(19) United States
(12) Patent Application Publication
(10) Pub. No.: US 2011/0236877 A1
(43) Pub. Date: Sep. 29, 2011

(54) BIOSENSOR AND METHOD USING THE SAME TO PERFORM A BIOTEST

(76) Inventors: Da-Jeng YAO, Hsinchu City (TW); Hsu-Chao Hao, Hsinchu City (TW)

(21) Appl. No.: 12/748,002

(22) Filed: Mar. 26, 2010

(51) Int. Cl. C12Q 1/00; C12M 1/34

(52) U.S. Cl. 435/4; 435/287.1

(57) ABSTRACT

The present invention discloses a biosensor and a method using the same to perform a biotest. The biosensor of the present invention integrates a SAW (Surface Acoustic Wave) device and a test polymer to detect an analyte in a liquid specimen. The test polymer reacts with the analyte. After reaction, the substantial weight decrease of the test polymer causes the variation of the SAW characteristics. The attributes and content of the analyte is detected and determined according to the variation of the SAW characteristics.

### FIELD OF THE INVENTION

The present invention relates to a biosensor, particularly to a biosensor which uses surface acoustic wave (SAW) to test a liquid specimen. The present invention also relates to a method using the abovementioned biosensor to perform a bio-test, particularly to a method which analyzes the SAW variation caused by the weight decrease of the test polymer.

### BACKGROUND OF THE INVENTION

General biological samples or specimens are mainly tested with two methods. In one of the methods, extraction, concentration and deposition processes or mass spectrographic analysis and chromatography are used to separate and purify a biological sample or specimen, and then the sample or specimen is tested. In the other method, a specific reaction is used to directly assay an analyte in a complicated sample. For example, a biochip or sensor is used to assay whether an analyte exists.

A biosensor generally comprises an identification material, a transducer and a signal processor. When the identification material reacts with the analyte the transducer transforms the resultant physical or chemical variation into an electric signal. The signal processor analyzes the electric signal and obtains a test result. In order to get an accurate test result, the identification material must have specificity corresponding to the analyte. The common-seen identification materials include biological tissues, microbes, organelles, cell receptors, enzymes, antigens and antibodies.

Refer to FIG. 1 for a conventional SAW (Surface Acoustic Wave) device. The conventional SAW device 1 comprises a piezoelectric substrate 11, a first transducer 12, a second transducer 13 and an external circuit (not shown in the drawing), wherein the first and second transducers 12 and 13 are respectively arranged at two sides of the surface of the piezoelectric substrate 11. Each of the transducers 12 and 13 has a plurality of InterDigital Transducers (IDTs) 14. When the external circuit applies an electric signal to the first transducer 12, the first transducer 12 transforms the electric signal into a surface acoustic wave via the piezoelectric effect. The surface acoustic wave is transmitted along the surface of the piezoelectric substrate 11 to the IDTs 14 of the second transducer 13. The second transducer 13 transforms the surface acoustic wave into the electric signal and outputs the electric signal via the external circuit. The transmission of the surface acoustic wave is easily to be influenced by the external environment. Therefore, the surface acoustic wave is very suitable to perform a test. The SAW device is compact, lightweight, low-cost and can be mass produced with a semiconductor technology. Further, the SAW device is very sensitive and has a high signal/noise ratio. Therefore, the SAW devices have been used in biosensors in recent years.

A R.O.C. publication No. 200804807 discloses a “Sensor for Detecting a Chemical or Biological Material”, wherein a biological label molecule is mixed with a sol-gel derivative to detect an analyte in a gas. An U.S. Pat. No. 5,658,732 discloses an “Assay Method for Biological Target Complexes on the Surface of a Biosensor”. An U.S. Pat. No. 5,814,525 discloses a “Piezoelectric Biosensor with a Ladder Polymer Substrate Coating”. These prior arts respectively disclose a piezoelectric biosensor and the method using the same to detect a biological specimen. They use a specific identification material to capture an analyte. The identification material has active binding sites to bond or couple with the analyte, whereby the analyte is detected.

Distinct from the abovementioned prior arts, the present invention proposes a biosensor which integrates a SAW device and a test polymer, and which detects the analyte according to the fact that the weight of the test polymer is substantially decreased by the reaction between the test polymer and the analyte.

### SUMMARY OF THE INVENTION

One objective of the present invention is to provide a biosensor and a method using the same to perform a bio-test, whereby an analyte in a liquid specimen can be fast and sensitively detected.

To achieve the abovementioned objective, the present invention proposes a biosensor which integrates a SAW (Surface Acoustic Wave) device and a test polymer comprises a piezoelectric substrate, a first transducer, a second transducer and a test polymer. The test polymer is formed on the piezoelectric substrate and able to react with an analyte. The first and second transducers are formed on the piezoelectric substrate and respectively arranged at two sides of the test polymer. The first and second transducers are respectively defined to an output end and an input end of the surface acoustic wave. The present invention also proposes a method using the abovementioned biosensor to perform a biotest, wherein the test polymer reacts with the analyte and loses weight, such that the characteristics of the SAW are changed by the weight decrease of the test polymer. The biosensor detects and analyzes the variation of the SAW characteristics to determine the attributes and content of the analyte. The embodiments are described in detail in cooperation with the drawings to demonstrate the technical contents of the present invention.

### BRIEF DESCRIPTION OF THE DRAWINGS

The embodiments of the present invention are described in cooperation with the following drawings.

FIG. 1 is a perspective view schematically showing the appearance of a conventional SAW device;

FIG. 2 is a perspective view schematically showing the appearance of a biosensor according to one embodiment of the present invention;

FIGS. 3A-3C are diagrams schematically showing the steps of a method according to one embodiment of the present invention; and

FIG. 4 is a diagram showing the results of experiments for detecting enzyme through a biosensor according to one embodiment of the present invention.

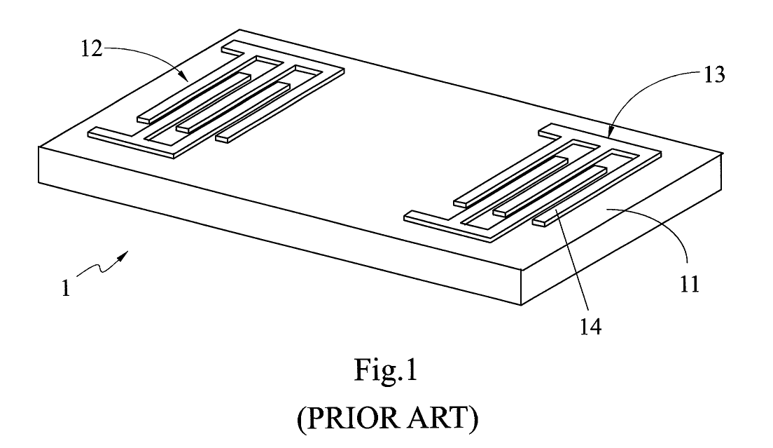

### DETAILED DESCRIPTION OF THE PREFERRED EMBODIMENTS

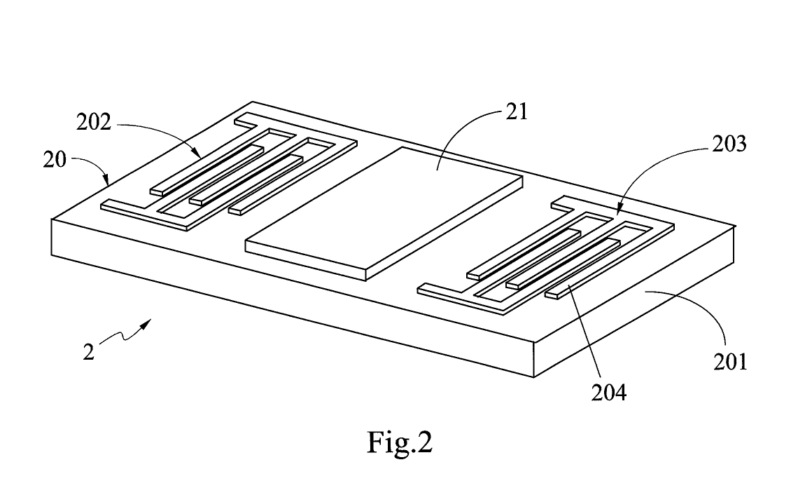

Below, the biosensor and the method using the same to perform a biotest are described simultaneously in accompany with the drawings to make easily understood the technical contents of the present invention. Refer to FIG. 2 for a perspective view schematically showing the appearance of a biosensor according to one embodiment of the present invention. The biosensor 2 of the present invention integrates a SAW (Surface Acoustic Wave) device 20 and a test polymer 21 to assay an analyte in a liquid specimen. The biosensor 2 of the present invention comprises a piezoelectric substrate 201, a first transducer 202, a second transducer 203, a test polymer 21 and an external circuit (not shown in the drawings). The test polymer 21 is formed on one side of the piezoelectric substrate 201 and able to react with a specific analyte. The term “specific” will be further explained later. The first and second transducers 202 and 203 are formed on the identical side of the piezoelectric substrate 201 where the test polymer 21 is formed and the first and second transducers 202 and 203 are respectively arranged at two sides of the test polymer 21. The first and second transducers 202 and 203 are respectively electrically connected to the external circuit. Each of the first and second transducers 202 and 203 has a plurality of interdigital transducers (IDTs) 204. The first transducer 202 receives an electric signal from the external circuit and transforms the electric signal into SAW. The SAW passes the test polymer 21 that has reacted, and the test polymer 21 changes the characteristics of the SAW. The second transducer 203 receives the SAW and transforms the SAW into an electric signal. Analyzing and comparing the electric signal can determine whether the analyte exists or implement the qualitative and quantitative analyses of the analyte.

In the abovementioned embodiment, the SAW device 20 is a shear horizontal SAW (SH-SAW for short) device. The piezoelectric substrate 201 is made of quartz, or lithium tantalate (LiTaO₃).

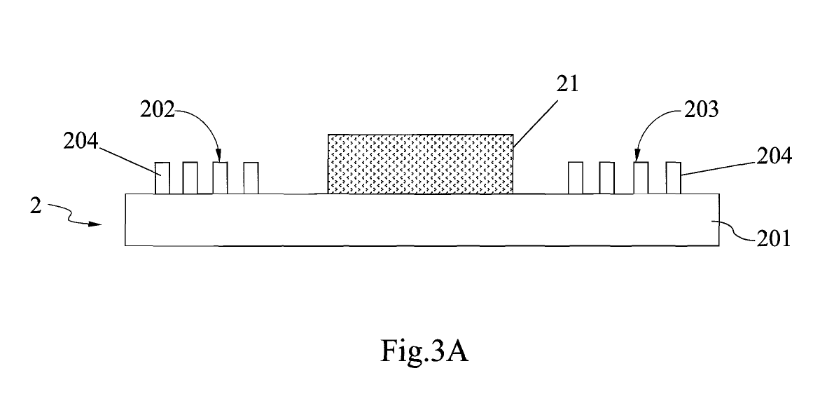

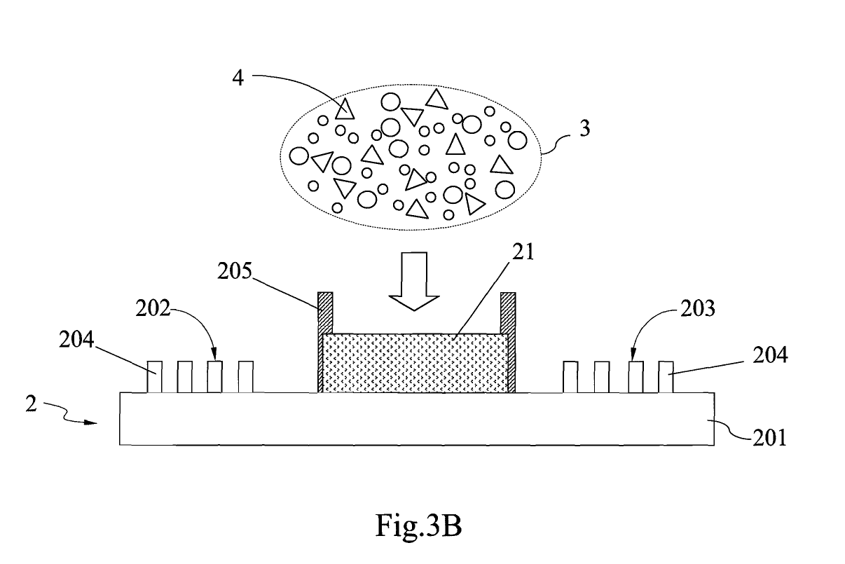

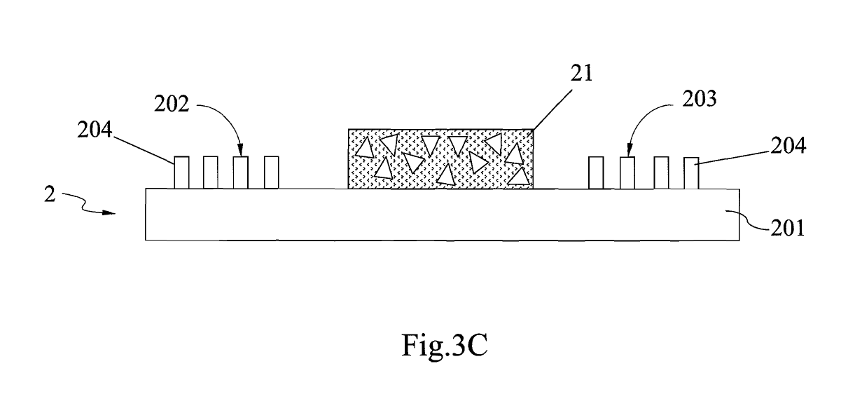

Refer to FIGS. 3A-3C for diagrams schematically showing the steps of a method according to one embodiment of the present invention. Firstly is prepared a biosensor 2 (as shown in FIG. 3A) corresponding to an analyte 4 (as shown in FIG. 3B), wherein the test polymer 21 is corresponding to the analyte 4. Herein, the term “corresponding” means that the test polymer 21 can detect the analyte 4. Next, an appropriate amount of liquid specimen 3 is taken to react with the test polymer 21 (as shown in FIG. 3B). In one embodiment, an accommodation space 205 is formed on the test polymer 21 of the biosensor 2, and the liquid specimen 3 is held in the accommodation space 205 so that the analyte 4 can react with the test polymer 21 thereinside. Then, the liquid specimen 3 is removed (washed or flushed away). After the test polymer 21 reacts with the analyte 4, the weight of the test polymer 21 substantially decreases, as shown in FIG. 3C, and even the electric conductivity or viscosity of the liquid specimen 3 may be changed. Thus, the central frequency and phase of the SAW passing through the test polymer 21 are also changed, or the energy of the SAW is reduced. Thus, analyzing the variation mode of the SAW can determine whether the liquid specimen 3 contains the analyte 4 and can implement the qualitative and quantitative analyses of the analyte 4.

It should be further explained that the substantial weight decrease of the test polymer 21 means the decrease of the physical dimensions, such as the decrease of the volume, area or thickness of the test polymer 21, which results from the reaction between the test polymer 21 and the analyte 4 and results in the decrease of mass or density of the test polymer 21. Thus are varied the physical characteristics of the SAW passing through the test polymer 21. The test polymer 21 has specificity to the analyte 4. “Specificity” means that the test polymer 21 cannot react with any material in the liquid specimen 3 except the analyte 4. In another embodiment, water, normal saline or buffer solution that does not react with the test polymer 21 is used to flush the test polymer 21 to remove the liquid specimen 3; then the residual humidity is dried with a baking or air-blowing way without damaging the test polymer 21. In yet another embodiment, the test polymer 21 is a solid-state material.

The biosensor and the method using the same to perform a biotest of the present invention can fast detect the attributes and content of the analyte 4 and thus can aid disease diagnosis and contribute to biomedicine researches. In the conventional technology, a captured molecule or material is formed on a film to capture a specific analyte. For example, an antigen is used to capture an antibody. The present invention is distinct from the conventional technology in that the test polymer 21 reacts with the analyte 4 in the liquid specimen 3, and the analyte 4 is detected via detecting the variation of the SAW resulting from the decrease of the physical quantities of the test polymer 21.

In one embodiment, the present invention is applied to detect an enzyme. In this embodiment, the test polymer 21 is a gelatin or a hydrogel, which can react with the enzyme and is coated on the piezoelectric substrate 201. The gelatin or hydrogel is a solid-state material at or below the normal atmospheric temperature. When the polymer (such as gelatin, hydrogel and the like) reacts with the enzyme (such as a proteolytic enzyme or a gelatin enzyme), hydrolysis occurs. Then, a portion of the gelatin or hydrogel is decomposed into amino acids and liquefied into a liquid. The liquid product of hydrolysis is removed (washed or flushed away) together with the liquid specimen 3. Therefore, the specificity of the gelatin or hydrogel can be used to detect an enzyme-contained liquid specimen 3 in the present invention.

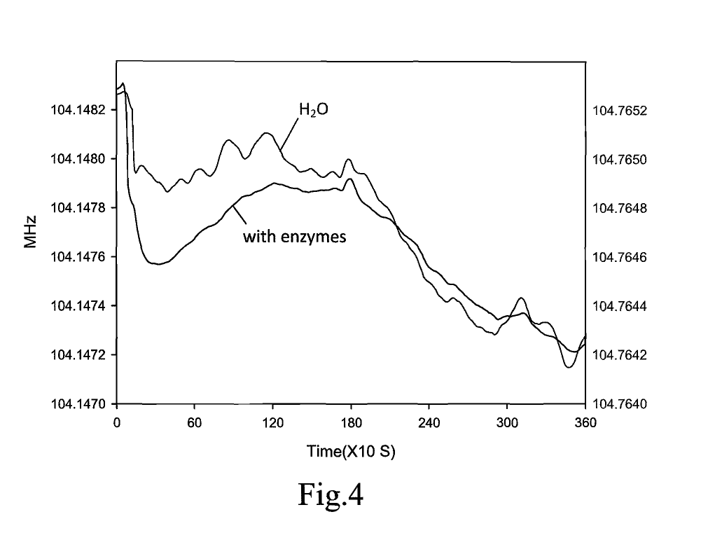

In one embodiment, the present invention is applied to detect the cancer of the urinary bladder or the diseases of the urinary system, wherein the liquid specimen 3 is urine. Normal urine (liquid specimen 3) contains none or only a trace of enzyme. Therefore, when the biosensor 2 detects normal urine, the change of the SAW characteristics is not obvious. When the bladder has a cancer or other diseases, the enzyme (analyte 4) is increased greatly. The great amount of the enzyme causes an obvious decrease of the physical quantity (mass) of the gelatin (test polymer 21) after reaction. Therefore, the present invention can be used to detect a urinary disease or cancer. Refer to FIG. 4 for a diagram schematically showing the results of experiments for detecting the enzyme through the biosensor 2 according to one embodiment of the present invention. In the experimental group, a solution containing the enzyme is used to simulate the liquid specimen 3. In the control group, water is used to simulate the liquid specimen 3. During the 1800 seconds of the reaction between the liquid specimen 3 and the test polymer 21, the frequency of the SAW has a significant difference. Therefore, the liquid specimen 3 containing the enzyme can be easily recognized. The decreased quantity of the test polymer 21 correlates with the characteristics of the SAW variation. Therefore, the present invention can be used to perform the quantitative analysis of the enzyme and to achieve the diagnosis and judgment of a disease.

The embodiments described above are only to exemplify the present invention but not to limit the scope of the present invention. Any equivalent modification or variation according to the technical contents disclosed in the specification and drawings of the present invention is to be also included within the scope of the present invention.

What is claimed is:

1. A biosensor, used to detect an analyte in a liquid specimen, comprising:
a piezoelectric substrate;
a test polymer formed on the piezoelectric substrate; and
a first transducer and a second transducer formed on an identical side of the piezoelectric substrate, and respectively arranged at two sides of the test polymer, and respectively defined to be an output end and an input end of a surface acoustic wave;
wherein the test polymer reacts with the analyte, characteristics of the surface acoustic wave are changed by a substantial weight decrease of the test polymer to detect the existence of the analyte.

2. The biosensor according to claim 1, wherein the piezoelectric substrate is made of quartz, or lithium tantalate (LiTaO₃).

3. The biosensor according to claim 1 further comprising an accommodation space for holding the liquid specimen.

4. The biosensor according to claim 1, wherein the analyte is an enzyme, and the test polymer is hydrolyzed.

5. The biosensor according to claim 1, wherein the surface acoustic wave is a shear horizontal surface acoustic wave.

6. A method for performing a biotest, used to detect an analyte in a liquid specimen, comprising the steps of:
preparing a surface acoustic wave biosensor including a test polymer corresponding to the analyte;
taking an appropriate amount of the liquid specimen to react with the test polymer;
removing the liquid specimen; and
detecting a surface acoustic wave passing through a surface of the surface acoustic wave biosensor and assaying the analyte via detecting a variation of characteristics of the surface acoustic wave caused by a substantial weight decrease of the test polymer.

7. A method for performing a biotest according to claim 6 used to detect an enzyme in the liquid specimen, wherein the test polymer is a gelatin.

8. A method for performing a biotest according to claim 6, wherein a buffer solution which does not react with the test polymer is used to wash the test polymer and remove the liquid specimen.

9. A method for performing a biotest according to claim 8, wherein the buffer solution is water or a normal saline.

10. A method for performing a biotest according to claim 8 further comprising a step of dehumidification after washing the test polymer with the buffer solution.
